(19) United States
(12) Patent Application Publication
(10) Pub. No.: US 2026/0294470 A1
(43) Pub. Date: Oct. 1, 2026

(54) HAIR IMPLANTER DEVICE

(71) Applicant: SAÚDE VIÁVEL S.A., E PORTO (PT)

(72) Inventors: Carlos Jorge PAULO PORTINHA, PORTO (PT); Tiago José ARIEIRA ESTEVES, PORTO (PT); João Marcelo CASANOVA ALMEIDA TOMÉ SANTOS, PORTO (PT); Ana Sofia SILVEIRA RIBEIRO, PORTO (PT); Pedro FIDALGO MORAIS, PORTO (PT); Diogo José DA COSTA GONÇALVES, PORTO (PT); João Pedro CORREIA LEITE, PORTO (PT)

(21) Appl. No.: 19/477,906

(22) PCT Filed: Mar. 27, 2024
(86) PCT No.: PCT/IB2024/052981

§ 371 Date: Oct. 23, 2025

(30) Foreign Application Priority Data
Apr. 24, 2023 (PT) 118617

(51) Int. Cl. A61B 17/34; A61B 17/00

(52) U.S. Cl. CPC A61B 17/3403; A61B 2017/00017; A61B 2017/00398; A61B 2017/00477; A61B 2017/00539; A61B 2017/00544; A61B 2017/00752; A61B 2017/3409

(57) ABSTRACT

A hair implanter device is characterized by comprising a set of actuators and a motor, wherein the set of actuators are configured to enable the movement of a needle and a mandrel from a first position to a second position, and a spring is mechanically adapted to ensure the movement of the needle and the mandrel from the second position to the first position; and wherein the motor enables the mechanical movement of a storage magazine between a set of predetermined positions.

### TECHNICAL FIELD

The present application describes a hair implanter device.

### Background Art

The hair implantation is a procedure performed by placing follicular units, one by one, in an implantation region of a patient. This is a very demanding and tiring procedure for the healthcare professional (HCP) who performs it, particularly during hair transplants, which might reach around 4000 follicular units required to implant. It also implies that the follicular units are handled several times during this procedure.

Focused on the existence of this problem, the present invention provides a novel medical device for the implantation phase of the hair transplant procedure in accordance with the medical devices' industry quality standards and GMP as defined by the European Union.

### SUMMARY

The present invention describes a hair implanter device characterized by comprising a set of actuators; and a motor; wherein the set of actuators are configured to enable the movement of a needle and a mandrel from a first position to a second position, and a spring is mechanically adapted to ensure the movement of the needle and the mandrel from the second position to the first position; and wherein the motor enables the mechanical movement of a storage magazine between a set of predetermined positions.

In a proposed embodiment of present invention, the set of actuators are selected from a pneumatic actuator, or a hydraulic actuator, or an electromechanical actuator, or a manual actuator, or combinations thereof.

Yet in another proposed embodiment of present invention, the set of actuators are connected to an external control device by means of a control connection selected from a control hose, or an electrical control cable, or a data control cable, or a mechanical control cable, or combinations thereof.

Yet in another proposed embodiment of present invention, the control hoses of the control connection are connected to the external control device and are fitted to the pneumatic actuators of the set of actuators by means of pneumatic connectors to ensure pressurized air contained in said hoses to activate said set of actuators.

Yet in another proposed embodiment of present invention, the set of actuators comprise a needle actuator and a mandrel actuator, the needle actuator being adapted to enable the deployment and/or retraction of the needle and the mandrel actuator being adapted to enable the deployment and/or retraction of the mandrel.

Yet in another proposed embodiment of present invention, the motor (8) is mechanically adapted to promote the movement of a magazine holder by means of a gearbox.

Yet in another proposed embodiment of present invention, a magazine coupler is mechanically configured to enable the coupling of the storage magazine thereto by means of a groove comprised in said magazine.

Yet in another proposed embodiment of present invention, the magazine holder is configured to transmit to the storage magazine positioning movements between the set of predetermined positions from an initial position to a following position.

Yet in another proposed embodiment of present invention, the set of predetermined positions comprise a distance between a set of storage holes comprised in the storage magazine and allow an alignment between said holes, the needle and the mandrel.

Yet in another proposed embodiment of present invention, the storage holes comprise a set of follicular units of patient.

Yet in another proposed embodiment of present invention, the magazine coupler is both mechanically adapted to the spring and to the needle actuator.

Yet in another proposed embodiment of present invention, the mandrel, during the movement from the first position to the second position, enables a movement of a follicular unit of the set of follicular units of the patient comprised in the storage hole compartments to a tip of the needle positioned in the second position.

The present invention also describes the operation method of the hair implanter device according to any of the previous claims comprising the steps of: a control connection activating a first actuator; the activated first actuator enabling the movement of the needle from a first position to a second position, exposing it in an extremity of the structure of the hair implanter device; a control connection activating a second actuator; the activated second actuator enabling the movement of the mandrel from a first position to a second position, said movement pushing a follicular unit comprised in a storage hole of the set of storage holes to a tip of the exposed needle; the first actuator being retracted, by means of the control connection, and being mechanically aided by the spring, enabling the movement of the needle from the second position to the first position, retracting it in the structure of the hair implanter device; the second actuator being retracted, by means of the control connection, and mechanically aided by the spring, enabling the movement of the mandrel from the second position to the first position; the motor enabling the mechanical movement of a storage magazine between a set of predetermined positions.

### GENERAL DESCRIPTION

The present invention discloses a medical device for the implantation phase of the hair transplant procedure.

The herein disclosed hair implanter device is a handheld device that can be used by a healthcare professional (HCP) to implant follicular units in a patient by combining the process of creating an incision on the skin surface and placing a follicular unit in that incision, in the dermis layer. The proposed process can be repeated for multiple times without the need to fully stop the hair implanter since the tool comprises a storage magazine with several pre-charged follicular units. To be noted that, in light of the applicant's extensive knowledge in this area, there are currently no automatized mechanical tools developed that encompass this technical feature that allows to implant multiple hair follicles, these being housed in a storage magazine, which in one of the herein disclosed embodiments, can be a rotating magazine. So, and for the proposed procedure, the herein disclosed hair implanter device is able to provide and deploy multiple follicular units on a patient between reloads of the storage magazine, as opposed to the tools known to date, which need to be completely replaced by the HCP every time a follicular unit is deployed in the patient, which substantially increases the number of repetitive movements performed by this HCP. The proposed device leads to an increasing speed of implantation of follicular units on patients, reducing surgery times, reducing the number of repetitive movements from the HCP, minimizing the handling exposure of the follicular units while increasing their survival rate.

The developed hair implanter device will enable the HCP to experience a less tiring and easier to use procedure when compared to the actual existing devices, while making said procedure less tiring and quicker for the patient. Additional advantages of using this device are related with the reduction of the time needed to perform the implantation of the follicular unit on the patient, which also lead to a reduction of the required levels of anaesthesia applied to the patients which is by far beneficial for their health.

Based on the above-mentioned problems, the developed hair implanter device also provides ergonomic handheld features that allow the HCP to be able of implanting multiple follicular units, one at a time, without the need to fully change the hair implanter device between each follicle placement in the patient. The technical features of the hair implanter device allow to reduce the implantation time, and particularly the number of movements executed by the HCP.

For example, and for illustrative purposes only, a typical implant procedure may take around 4000 implant movements on the skin surface of the patient by the HCP. This will include 4000 implant movements based on creating an incision on the skin surface of a patient and placing an independent follicular unit in that incision. On the illustrated implant procedure, it would be required the replacement of the used hair implanter device by a new hair implanter device comprising a fresh new follicular unit set to be placed in next incision in the skin surface of the patient, particularly until reaching the dermis layer, being this operation repeated at each skin incision. This typical procedure would be repeated 4000 times, related to the 4000 implant movements required to promote the placement of 4000 follicular units in the skin surface of the patient, in particular, in the dermis layer of the patient. With the proposed hair implanter device, and in one of the proposed embodiments herein disclosed for illustrative purposes, the previous procedure can be reduced from, for example, 4000 to 160 implant movements, being this enabled by the improvement of the replacement procedure of the hair implanter devices by a device that comprises a magazine, that may store multiple follicular units at a time (at one of the proposed embodiments at least 25 in a cylindrical shaped magazine), instead of singular follicular units.

Based on the above, the hair implanter device provides an innovative solution to perform the implantation process in less time and more quality than current existing technologies.

The hair implanter device, as previously mentioned, is controlled by a control unit which has its own Design and Development Dossier, as well as the storage magazine, which is a key component of the follicular unit loading device of the hair implanter device.

### BRIEF DESCRIPTION OF THE DRAWINGS

For better understanding of the present application, figures representing preferred embodiments are herein attached which, however, are not intended to limit the technique disclosed herein.

FIG. 1—discloses an illustration of the hair implanter device (100), where the reference numbers relate to:
1—Magazine holder;
2—Motor cover;
4—Magazine coupler;
6—Magazine coupler O-Ring;
7—Gear box;
8—Motor;
9—Actuators support;
10—Motor cover stopper;
11—Needle actuator;
14—Mandrel actuator;
16—Cable fixer;
17—Front Cover;
18—Back cover;
19—Storage Magazine;
20—Pointer;
21—Needle;
22—Needle hub;
23—First actuator connector;
24—Mandrel;
25—Mandrel hub;
26—Spring;
27—Placement device;
29—Second actuator connector.

FIG. 2—discloses an illustration of the method of operation of the control unit (200), where the reference numbers relate to:
201—user interface touch screen;
202—micro-controller;
203—compressed air;
19—Storage magazine;
205—motor controller;
206—solenoid valves;
100—Hair implanter device.

FIG. 3—-discloses an illustration of the storage magazine (19) where it is depicted the positioning of the storage holes (19.1) in the inner rim of the storage magazine (19), in one of the proposed embodiments, with a cylindrical shape, and the rotational groove (19.2) that ensures the mechanical fit to promote the rotation of the storage magazine (19).

FIG. 4—discloses a cut-through illustration of the storage magazine (19) where it is possible to visualize the rotational groove (19.2) and the storage holes (19.1) that run through the drum of the magazine from a top side to another bottom side.

### DESCRIPTION OF EMBODIMENTS

With reference to the figures, some embodiments are now described in more detail, which are however not intended to limit the scope of the present application.

The hair implanter device (100), in one of the preferred embodiments of the disclosed invention, comprises two actuators (11, 14), a needle actuator (11) and a mandrel actuator (14). These actuators can be selected for a random combination of existing technologies which might include, for example, a pneumatic actuator, or a hydraulic actuator, or an electromechanical actuator, or a manual actuator, or other technically adapted and suitable for the purpose. The needle actuator (11) is responsible for enabling the deployment and retraction of the needle (21), which will penetrate the skin surface of a patient until reaching the dermis layer, and the mandrel actuator (14) is responsible for the deployment and retraction of the mandrel (24).

In one of the proposed embodiments of the present invention, a motor (8) and a gearbox (7) are also comprised in the device (100) and are configured to promote the movement of the storage magazine (19) between a set of predetermined positions, said positions being related with the distance between storage holes (19.1) where follicular units from the patient will be stored. In a particular embodiment of the invention, the storage magazine (19) comprises a cylindrical shape, and the motor (8) promotes the rotation of the magazine holder (1), that through mechanical and coupling means (4), transfers that rotation, promotion the movement of the storage magazine (19). For the proposed arrangement, the motor (8) can comprise a stepper motor. The storage magazine (19) is coupled to the device (100) by a mechanical coupling feature, or magazine coupler (4), that fits a rotational groove (19.2) comprised in the storage magazine (19). In the proposed embodiment, the groove (19.2) is located in the center of the storage magazine (19), but additional embodiments can also be foreseen without limiting the action and scope of the promoted movement over said storage magazine (19). The coupling and uncoupling mechanism (4) is also mechanically connected to a spring (26), as well as the needle actuator (11), configured to deploy and retract the needle (21). The storage magazine (19), in one of the preferred but not limiting embodiments of the present invention, comprises twenty-five storage hole-compartments (19.1) that will be filled with follicular units, for further implant in the patient. The device (100), as previously suggested as one of the possible embodiments, is actuated by a control pedal, not illustrated in the figures, which will enable the exposure of the needle (21), and which will be set on the skin surface of the patient. The mandrel (24) is configured to move the follicular units comprised in the storage hole compartments (19.1) of the storage magazine (19) to the tip of the exposed needle (21) and set it on the skin surface of the patient. In one of the proposed embodiments, the needle (21) comprises a hollow interior, being similar to a hollow tube, but with adequate dimensions to perform hair implant procedures.

The implantation procedure consists in loading the follicular units, located into the storage holes (19.1) of the storage magazine (19), which is comprised, in one of the preferred embodiments, in the vicinity and aligned with the needle (21), into the skin of the patient. As previously suggested, the preferred embodiment of the disclosed storage magazine (19) comprises an arrangement of 25 storage holes (19.1) that will be filled with the follicular units, which will be further loaded it into the implanter device (100) and deposited into the skin surface of the patient. In an initial stage, before pressing the control means, both the actuators, i.e., the needle actuator (11) and the mandrel actuator (14) will be in a retracted position. The loaded hair implanter device (100) with the 25 follicular units, is firstly actuated by means of a control pedal, a button, or a fully automatic actuation system with a configured velocity of actuation, which will enable the exposure of the needle (21), which will be further placed (or pushed) into the skin surface of the patient by the HCP. When the actuation means are pressed, the needle actuator (11) is activated, moving towards the needle (21), pushing it in a forward movement to an external positioning with regard to the device (100); while the mandrel actuator (14) remains in the same retracted position. A second actuation instruction from the HCP provided by the actuation means, or a particular configured button for this effect as previously suggested, will activate the mandrel actuator (14) that will push the mandrel (24) in a forward movement that will promote displacement of the follicular unit from the storage magazine (19) to the tip of the exposed needle (21) which is inserted into the skin surface of the patient, keeping said follicular unit inside the needle (21). In an automatic sequence, without any further actuation for the HCP, the needle (21) retracts, due to a retraction movement of the needle actuator (11) that retrieves to the initial position, keeping the mandrel (24) in place, while the follicular unit is maintained within the skin implantation location of the patient. After a predetermined time duration, the mandrel (24) will also retract, due to a retraction movement of the mandrel actuator (11) that retrieves to the same initial position, leaving the follicular unit in the correct implantation location of the patient, and, to finish the implant cycle, the storage magazine (19) will move to the next hole (19.1) comprising follicular units, where, in the illustrated example, rotates to the next hole (19.1) of the storage magazine (19); this step being ensured by the rotational movement provided by the motor (8) to the magazine coupler (4). The storage magazine (19) structural shape herein illustrated may have constructive and functional variations that, in some manner, may use the same operational concept of the overall disclosed device (100). The follicular unit will travel inside a capillary tube, or needle, during the normal implantation procedure on the patient. The actuation of the implanter (100) is going to be done by a pedal, a button, or a fully automatic actuation with a configured velocity of actuation which is controlled by the HCP.

Within the implant procedure, and particularly before it, in a preparation stage, the storage magazine (19) is primarily filled by the HCP, or by a healthcare assistant, in a hand or fully automated procedure, resorting to the use of auxiliary equipment particularly developed to ensure the loading of the follicle units in the storage magazine (19).

Other pre-defined requirements for the hair implanter device (100) regard that it should comprise diameter between 9 mm and 50 mm, and, in optimal a setting, should comprise a maximum diameter of 28 mm. This will enable optimal performance in terms of manoeuvrability and hand adequacy for the HCP. The same applies to the length of the device (100) that should have comprise a length between 50 mm and 250 mm, and, in an optimal embodiment should be inferior than 210 mm. The weight of the device (100) should also be comprised between 50 grams and 500 grams, and, in optimal a setting, should be inferior to 150 grams.

The used materials and parts of the hair implanter device (100) must fulfil all the clinical regulations, particularly the ones which are in contact with the patient's body which have to be biocompatible. Cross-contamination must be avoided, based on the material itself of which the components are manufactured, or due to difficulty or impossibility to clean and disinfect said components. Damage to the follicle during the handling inside the device must also be ensured to not happen. The hair implanter device (100), like illustrated in FIG. 1, is configured to ensure safety measures and to limit to the maximum extent of an occurrence of harm by the clinical personal to the patient. This concern is related with the needle (21) that comprises a specific placement device (27) which comprises a disposable chamber that isolates the needle (21). After the clinical usage, both chamber (27) and needle (21) are disposed of. The pointer (20) of the implanter device (100) also comprises an additional placement adapter (27) to promote its placement and removal due to the proximity to the needle (21). In this way, both the needle (21) and the pointer (20), resort to the use of these particular tools placement devices (27), which help in the assembly and disassembly of said components (20, 21) in the device (100). The use of the adapters (27) is due to the fact that they are sharp cutting tools that are in permanent contact with blood, and their use in this clinical procedure ensures compliance with additional safety and health measures. Further attention was directed during the development to ensure that the electric wiring is considered to be isolated from the exterior of the implanter device (100). In one of the preferred embodiments of the present invention, the compressed air used to move the actuators (11, 14) also operates in an independent circuit which is isolated from the patient, thus preventing the possibility of contamination due to contact or tool sharing. Additional security features are also included within the implanter device (100) which comprise a motor cover (2) and magazine coupler O-Ring (6), to prevent fluids and other elements to pass through the patient to the device (100), or vice-versa. In line with the previous description, a motor cover stopper (10) is mechanically adapted to the motor cover (2), which movement will be enabled by the needle actuator (11), which is kept positionally aligned within a movement range by means of a actuator support (9); the same occurring with the mandrel actuator (14).

Additional constructional features of the device (100) are illustrated in FIG. 1, as the front and back covers (17, 18) and cable fixer (16), that provide support nor only for the assembly of the device (100), but also for the ergonomics and security of it.

The pointer (20) is used to allow the HCP to place and adjust the hair implanter device (100) within a desired orientation and positioning with regard to the patient before the follicular units are therein inserted. It allows for a better and more natural follicular unit implant and is mechanically adapted in the surroundings of the needle (21). The pointer (20) improves the precision and alignment accuracy of the needle (21) during the follicular unit insertion in the dermis layer of the patient. In one of the proposed embodiments (the invention, the pointer might comprise one of a pointed shaped object, a laser guide or a light emitting source. The needle hub (22) ensures the mechanical connection between the hair implanter device (100) and the needle (21), allowing for an improvement in the placement and removal procedure of said needle (21), so to perform its correct and adequate sterilization. The first actuator connector (23) enables the connection of the hair implanter device (100) to a first control connection, for example an external hose, that first hose being further connected to a control device for the control and supply of pneumatic/pressurized air to the needle actuator (11), which enables its movement when required and instructed by said external control device. In a similar manner, the second actuator connector (29) enables the connection of the hair implanter device (100) to a second control connection, for example an external hose, that second hose being further connected to the control device for the control and supply of pneumatic/pressurized air to the mandrel actuator (14), which enables its movement when required and instructed by said external control device. The Mandrel hub (25) enables the mechanical connection and physical adaptation between the mandrel (24) and the hair implanter device (100), providing a solid and easy to place and remove feature for this component for sterilization purposes.

The spring (26) enables the retraction of all the moving parts back to the original position inside the hair implanter device (100) after the follicle procedure deployment into the skin surface of the patient occurs. Based on the previous suggested embodiment, when the air pressure is removed from the pressurized air hoses that supply the needle actuator (11) and the mandrel actuator (14), which are thereto attached by means of the first actuator connector (23) and the second actuator connector (29), the spring will enable the retraction of said elements by means of the spring mechanical force pushes everything back to the original position. In short, while the air pressure is supplied to the hoses, the pushing of the actuators (11, 14) in a forward particular action moment occurs, overcoming the mechanical retraction force of the spring (26). When the air pressure is removed from the hoses, the spring (26) pushes all the remaining parts back to the initial position (because they no longer have the air pressure force to counteract it).

In terms of overall performance of the implanter device (100), it depends on the operating HCP; however, the implantation procedure is expected to ensure the placement of a hair follicle every 5 seconds. In addition, because of the larger quantity of follicular units available in the tool, to be implanted at once without the need of extra movements to pick follicular units, the overall performance will be increased in comparison with the currently existing solutions, one follicle at a time. It will also result in a decreased procedure time for the patient itself.

As illustrated in FIG. 2, the hair implanter device (100) will be operated by a control unit (200) that, preferably, will comprise a user interface touch screen (201) adapted to collect data inputs from the HCP, which will be further forwarded to a micro-controller (202) which is configured to centralize, control and command the overall tasks associated the implant procedures. The micro-controller (202) will not only control the operation of the overall system, with particular focus on the cylinder magazine (19), the motor (8) through the motor controller (205), and solenoid valves (206), but also a compressed air device (203) which provides compressed air to the device (100) by means of a set of hoses therein connected by means of the first actuator connector (23).

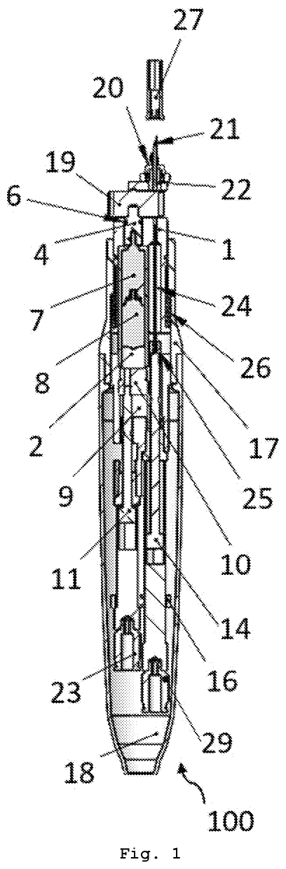

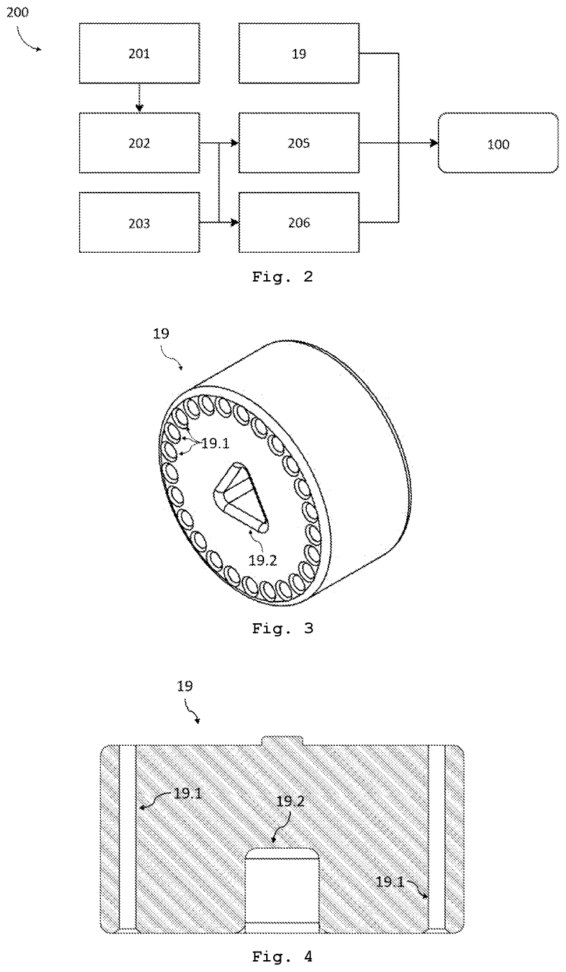

In FIG. 3 it is illustrated one possible embodiment of the magazine (19), here in a cylindrical shape, where it is depicted the positioning of the storage holes (19.1) which are positioned in the inner rim of the magazine (19), and also visible is the rotational groove (19.2) that ensures the mechanical fit to the magazine coupler (4) to further enable the rotation of the magazine (19). On FIG. 4 it is disclosed a cut-through illustration of the cylinder magazine (19) where it is possible to visualize the rotational groove (19.2) and the storage holes (19.1) that run through the drum of the magazine (19) from a top side to another bottom side.

1. A hair implanter device comprising
a set of actuators; and
a motor;
wherein the set of actuators are configured to enable the movement of a needle and a mandrel from a first position to a second position, and a spring is mechanically adapted to ensure the movement of the needle and the mandrel from the second position to the first position; and
wherein the motor enables the mechanical movement of a storage magazine between a set of predetermined positions.

2. The hair implanter device according to claim 1, wherein the set of actuators are selected from the group consisting of a pneumatic actuator, a hydraulic actuator, an electromechanical actuator, a manual actuator, and combinations thereof.

3. The hair implanter device according to claim 1, wherein the set of actuators are connected to an external control device by means of a control connection selected from the group consisting of a control hose, an electrical control cable, a data control cable, a mechanical control cable, combinations thereof.

4. The hair implanter device according to claim 1, wherein the control hoses of the control connection are connected to the external control device and are fitted to the pneumatic actuators of the set of actuators by means of pneumatic connectors to ensure pressurized air contained in said hoses to activate said set of actuators.

5. The hair implanter device according to claim 1, wherein the set of actuators comprise a needle actuator and a mandrel actuator, the needle actuator being adapted to enable the deployment and/or retraction of the needle and the mandrel actuator being adapted to enable the deployment and/or retraction of the mandrel.

6. The hair implanter device according to claim 5, wherein the motor is mechanically adapted to promote the movement of a magazine holder by means of a gearbox.

7. The hair implanter device according to claim 1, wherein a magazine coupler is mechanically configured to enable the coupling of the storage magazine thereto by means of a groove comprised in said magazine.

8. The hair implanter device according to claim 6, wherein the magazine holder is configured to transmit to a storage magazine positioning movements between a set of predetermined positions from an initial position to a following position.

9. The hair implanter device according to claim 8, wherein the set of predetermined positions comprise a distance between a set of storage holes comprised in the storage magazine and allow an alignment between said set of storage holes (19.1), the needle and the mandrel.

10. The hair implanter device according to claim 9, wherein the storage holes comprise a set of follicular units of a patient.

11. The hair implanter device according to claim 7, wherein the magazine coupler is both mechanically adapted to the spring and to the needle actuator.

12. The hair implanter device according to claim 9, wherein the mandrel, during the movement from the first position to the second position, enables a movement of a follicular unit of the set of follicular units of the patient comprised in the storage hole compartments to a tip of the needle positioned in the second position.

13. An operation method of the hair implanter device according to claim 1, comprising the steps of:
activating a first actuator, by means of a control connection, wherein the activated first actuator enables the movement of the needle from a first position to a second position, exposing the needle in an extremity of the structure of the hair implanter device;
activating a second actuator, by means of the control connection, wherein the activated second actuator enables movement of the mandrel from a first position to a second position, said movement pushing a follicular unit comprised in a storage hole of the set of storage holes to a tip of the exposed needle;
retracting the first actuator, by means of the control connection, and being mechanically aided by the spring, enabling the movement of the needle from the second position to the first position, retracting the in the structure of the hair implanter device;
retracting the second actuator, by means of the control connection, and mechanically aided by the spring, enabling the movement of the mandrel from the second position to the first position;
mechanically moving a storage magazine, via the motor, between a set of predetermined positions.
